(19) United States
(12) Patent Application Publication
(10) Pub. No.: US 2021/0137807 A1
(43) Pub. Date: May 13, 2021

(54) COSMETIC COMPOSITION COMPRISING TWO SPECIFIC CATIONIC SURFACTANTS AND A SILICONE EMULSION, AND A COSMETIC TREATMENT PROCESS

(71) Applicant: L'OREAL, Paris (FR)

(72) Inventors: Claire-Marie LESAULNIER, SAINT-OUEN (FR); Laurent CHESNEAU, SAINT-OUEN (FR)

(21) Appl. No.: 16/623,591

(22) PCT Filed: Jun. 21, 2018
(86) PCT No.: PCT/EP2018/066571

§ 371 Date: Dec. 17, 2019

(30) Foreign Application Priority Data
Jun. 21, 2017 (FR) 1755647

(51) Int. Cl. A61K 8/41; A61K 8/891; A61K 8/898; A61K 8/86; A61K 8/06; A61K 8/34; A61K 8/37; A61Q 5/12

(52) U.S. Cl. CPC A61K 8/416; A61K 8/891; A61K 8/898; A61K 8/86; A61K 8/062; A61K 2800/5426; A61K 8/37; A61Q 5/12; A61K 2800/21; A61K 2800/596; A61K 2800/5422; A61K 8/342

(57) ABSTRACT

The present invention relates to a cosmetic composition comprising:

(i) cationic surfactants of formula (Ia):

[chemical structure]

in which:

R5 and R6 represent a linear or branched, saturated or unsaturated C8-C30 aliphatic group optionally comprising heteroatoms; or else an aromatic group,

R7 and R8 represent a linear or branched C1-C6 aliphatic group, and

Y⁻ is an anion;

(ii) additional cationic surfactants, different from the cationic surfactants of formula (Ia), and

(iii) an oil-in-water emulsion having a particle size D50 of less than 350 nm, comprising a polydialkylsiloxane comprising trialkylsilyl end groups, an aminosilicone and a non-ionic surfactant having an HLB ranging from 10 to 16.

The invention also relates to a cosmetic process for treating, more particularly for conditioning, the hair.

The present invention relates to a cosmetic composition, in particular a hair composition, comprising the combination of at least two specific cationic surfactants and a silicone emulsion; and also to a cosmetic treatment process using this composition.

In the field of the cosmetic treatment of keratin fibres, in particular human keratin fibres, and in particular the hair, it is sought to treat keratin fibres that have been sensitized and/or damaged, in particular following various external attacks, such as mechanical attacks, for example linked to disentangling or blow drying, or else chemical attacks, for example linked to dyeing or permanent-waving.

These attacks can have repercussions on the quality of keratin fibres, and can in particular cause a degradation of the surface properties of keratin fibres, which become unsmooth and uneven at the surface, hence they are not as easy to disentangle and have a less pleasant feel, whether on dry hair or wet hair.

Care products exist which make it possible to limit these phenomena. These products generally contain one or more conditioning agents, such as cationic surfactants, fatty alcohols or silicones.

However, some of these conditioning agents have the drawback of thickening the texture of hair care products and of resulting in an excessively high viscosity of the compositions; some conditioning agents also have the drawback of depositing too much conditioning agent on the hair, which generally results in a softening or a lack of tonicity of the fibre, and/or a lack of resistance of the fibre, and also in a feeling of being unclean and greasy to the touch, due in particular to excessively rapid regreasing between two shampooing operations.

The applicant has now discovered that the combination of at least two specific cationic surfactants and of an emulsion of at least two silicones makes it possible to overcome the drawbacks set out above.

One subject of the invention is thus a cosmetic composition, in particular a hair composition, comprising: p (i) one or more cationic surfactants of formula (Ia):

[chemical structure]

in which:

R5 and R6, which may be identical or different, represent a linear or branched, saturated or unsaturated aliphatic group comprising from 8 to 30 carbon atoms, preferably from 12 to 24 carbon atoms, said aliphatic group possibly comprising one or more heteroatoms such as oxygen, nitrogen, sulfur and/or halogens; or an aromatic group such as an aryl or alkaryl group, in particular a benzyl group;

R7 and R8, which may be identical or different, represent a linear or branched aliphatic group comprising from 1 to 6 carbon atoms;

Y⁻ is an anion;

(ii) one or more cationic surfactants other than the cationic surfactants of formula (Ia), and preferably corresponding to formula (Ib) below:

[chemical structure]

in which:

R1 represents a linear or branched, saturated or unsaturated aliphatic group comprising from 8 to 30 carbon atoms, preferably from 12 to 24 carbon atoms, said aliphatic group possibly comprising one or more heteroatoms such as oxygen, nitrogen, sulfur and/or halogens;

R2 to R4, which may be identical or different, represent a linear or branched, saturated or unsaturated aliphatic group comprising from 1 to 6 carbon atoms;

X⁻ is an anion; and

(iii) an oil-in-water emulsion having a particle size D50 of less than 350 nm, and comprising:

a silicone mixture comprising (i) a polydialkylsiloxane comprising trialkylsilyl end groups, having a viscosity at 25° C. ranging from 40 000 to 100 000 mPa·s and (ii) an aminosilicone having a viscosity at 25° C. ranging from 1000 to 15 000 mPa·s and an amine number ranging from 2 to 10 mg of KOH per gram of aminosilicone;

a surfactant mixture comprising one or more non-ionic surfactants, said mixture having an HLB ranging from 10 to 16, and

water.

The composition according to the invention has good cosmetic properties, in particular conditioning cosmetic properties; it in particular makes it possible to obtain high performance levels in terms of visual smoothness and smoothness to the touch (smooth nature) of the hair, said smoothness being noticeable over the entire hair, that is to say from the root of the hair to the end thereof (root-end smoothness).

In addition, the composition according to the invention gives the keratin fibres tonicity.

It also makes it possible to obtain a non-lasting “cleanness” effect, that is to say that the hair has a non-greasy, natural feel which endures between two shampooing operations.

The composition according to the invention also makes it possible to deposit the required amount of conditioning agents to obtain very good performance levels, in particular in terms of smoothness, while at the same time avoiding excessive deposition which would be detrimental to the tonicity of the fibres and their clean feel.

It is particularly suitable for sensitized hair, in particular damaged, split, brittle hair, to which it can provide strength, vitality and body, said hair thus appearing revitalized, repaired and reinforced.

The composition according to the invention has, moreover, a viscosity suitable for easy application, without risk of it running. It has been noted that this viscosity changes little after a prolonged storage period.

Advantageously, the composition according to the invention has a viscosity of greater than 800 centipoises (cps), preferably greater than 900 cps, better still of between 900 and 1500 cps (viscosity measured using a Rheomat RM 200, at 25° C., spindle 3, 200 revolutions/min, measured at 30 s).

In the text hereinbelow, and unless otherwise indicated, the limits of a range of values are included in that range, in particular in the expressions “between” and “ranging from . . . to . . . ”.

The expression “at least one” used in the present description is equivalent to the expression “one or more”.

(i) Cationic surfactants of formula (Ia)

The composition according to the invention comprises one or more surfactants of formula (Ia) as defined above.

Preferably, R5 represents a benzyl group or a linear or branched, saturated aliphatic group comprising from 8 to 30 carbon atoms, preferably from 12 to 24 carbon atoms, said aliphatic group possibly comprising one or more heteroatoms such as oxygen and/or nitrogen.

Preferably, R6 represents a benzyl group or a linear or branched, saturated aliphatic group comprising from 8 to 30 carbon atoms, preferably from 12 to 24 carbon atoms, said aliphatic group possibly comprising one or more heteroatoms such as oxygen and/or nitrogen.

The aliphatic groups are for example chosen from C8-C30 alkyl, C8-C30 alkoxy, (C2-C6)polyoxyalkylene, C8-C30 alkylamide, (C12-C22)alkylamido(C2-C6)alkyl, (C12-C22)alkyl acetate and C8-C30 hydroxyalkyl groups.

In particular, R5 may be a saturated linear C8-C30, in particular C12-C24, alkyl group, or a (C12-C22)alkylamido(C2-C6)alkyl group. Preferentially, R5 may be a behenyl, cetyl, stearyl or stearamidopropyl radical.

In particular, R6 may be a saturated linear C8-C30, in particular C12-C24, alkyl group, or a (C12-C22)alkyl acetate group. Preferentially, R6 may be a benzyl, behenyl, cetyl, stearyl or myristyl acetate radical.

Preferably, R7 and R8, which may be identical or different, represent a saturated linear alkyl group comprising from 1 to 6 carbon atoms, in particular from 1 to 3 carbon atoms.

Preferentially, R7 and R8 represent a methyl group.

Preferably, Y⁻ is an anion chosen from halides such as chloride, bromide and iodide; phosphates, acetates, lactates, (C1-C4)alkyl sulfates, (C1-C4)alkyl- or (C1-C4)alkylarylsulfonates.

Preferably, the cationic surfactant(s) of formula (Ia) are chosen from di(C8-C30)alkyldimethylammonium salts, and in particular distearyldimethylammonium salts, dicetyldimethylammonium salts, benzyldimethylstearylammonium salts, stear-amidopropyldimethyl(myristyl acetate)ammonium salts, and mixtures thereof.

More particularly, they are chosen from di(C8-C30)alkyldimethylammonium halides, in particular chlorides, and in particular distearyldimethylammonium halides, in particular chlorides, dicetyldimethylammonium halides, in particular chlorides, benzyldimethylstearylammonium halides, in particular chlorides, stearamidopropyldimethyl(myristyl acetate)ammonium halides, in particular chlorides; or else from di(C8-C30)alkyldimethylammonium (C1-C4)alkyl sulfates, in particular methosulfates, and in particular distearyldimethylammonium (C1-C4)alkyl sulfates, in particular methosulfates, dicetyldimethylammonium(C1-C4)alkyl sulfates, in particular methosulfates, benzyldimethylstearylammonium (C1-C4)alkyl sulfates, in particular methosulfates, stearamidopropyldimethyl(myristyl acetate)ammonium (C1-C4)alkyl sulfates, in particular methosulfates; and also mixtures thereof.

Preferentially, the cationic surfactant(s) of formula (Ia) are chosen from dicetyldimethylammonium halides, in particular chlorides, or dicetyldimethylammonium (C1-C4)alkyl sulfates, in particular methosulfates.

The composition according to the invention preferably comprises the surfactant(s) of formula (Ia) in a total amount ranging from 0.1% to 10% by weight, better still from 0.2% to 10% by weight, preferentially from 0.5% to 5% by weight, relative to the total weight of the composition.

(ii) Additional Cationic Surfactants

The composition according to the also invention comprises one or more cationic surfactants different from the cationic surfactants of formula (Ia) as defined above.

The term “cationic surfactant” is intended to mean a surfactant that is positively charged when it is contained in the composition according to the invention. This surfactant may bear one or more positive permanent charges or may contain one or more cationizable functions in the composition according to the invention.

The additional cationic surfactant(s) (ii) are preferably chosen from primary, secondary or tertiary fatty amines, which are optionally polyoxyalkylenated, or salts thereof, and quaternary ammonium salts, and mixtures thereof.

The fatty amines generally comprise at least one C₈ to C₃₀ hydrocarbon-based chain.

Among the quaternary ammonium salts, mention may be made in particular of:

(a) the cationic surfactants of formula (Ib):

[chemical structure]

in which:

R1 represents a linear or branched, saturated or unsaturated aliphatic group comprising from 8 to 30 carbon atoms, preferably from 12 to 24 carbon atoms, said aliphatic group possibly comprising one or more heteroatoms such as oxygen, nitrogen, sulfur and/or halogens;

R2 to R4, which may be identical or different, represent a linear or branched, saturated or unsaturated aliphatic group comprising from 1 to 6 carbon atoms;

X⁻ is an anion.

Preferably, R1 represents a linear or branched, saturated aliphatic group comprising from 8 to 30 carbon atoms, preferably from 12 to 24 carbon atoms, said aliphatic group possibly comprising one or more heteroatoms such as oxygen and/or nitrogen.

The aliphatic groups are for example chosen from C8-C30 alkyl, C8-C30 alkoxy, (C2-C6)polyoxyalkylene, C8-C30 alkylamide, (C12-C22)alkylamido(C2-C6)alkyl, (C12-C22)alkyl acetate and C8-C30 hydroxyalkyl groups.

In particular, R1 may be a saturated linear C8-C30, in particular C12-C24, alkyl group, or a (C12-C22)alkylamido(C2-C6)alkyl group. Preferentially, R1 may be a behenyl, cetyl or palmitylamidopropyl radical.

Preferably, R2 to R4, which may be identical or different, represent a saturated linear alkyl group comprising from 1 to 6 carbon atoms, in particular from 1 to 3 carbon atoms. Preferentially, R2, R3 and R4 represent a methyl group.

Preferably, X⁻ is an anion chosen from halides such as chloride, bromide and iodide; phosphates, acetates, lactates, (C1-C4)alkyl sulfates, (C1-C4)alkyl- or (C1-C4)alkylarylsulfonates.

(b) quaternary ammonium salts of imidazoline, for instance those of formula (II) below:

[chemical structure]

in which:

R32 represents an alkenyl or alkyl group comprising from 8 to 30 carbon atoms, for example derived from tallow fatty acids,

R₃₃ represents a hydrogen atom, a C₁ to C₄ alkyl group or an alkenyl or alkyl group comprising from 8 to 30 carbon atoms,

R₃₄ represents a C₁ to C₄ alkyl group,

R₃₅ represents a hydrogen atom or a C₁ to C₄ alkyl group, and

X⁻ is an anion, in particular chosen from the group of halides, phosphates, acetates, lactates, alkyl sulfates, alkyl- or alkylarylsulfonates in which the alkyl and aryl groups preferably comprise, respectively, from 1 to 20 carbon atoms and from 6 to 30 carbon atoms.

Preferably, R₃₂ and R₃₃ denote a mixture of alkenyl or alkyl groups comprising from 12 to 21 carbon atoms, for example derived from tallow fatty acids, R₃₄ denotes a methyl group and R₃₅ denotes a hydrogen atom. Such a product is sold, for example, under the name Rewoquat® W 75 by Rewo;

(c) di- or triquaternary ammonium salts, in particular of formula (Ill):

[chemical structure]

in which

R₃₆ denotes an alkyl radical comprising approximately from 16 to 30 carbon atoms, which is optionally hydroxylated and/or interrupted with one or more oxygen atoms,

R₃₇ is chosen from hydrogen or an alkyl radical comprising from 1 to 4 carbon atoms or a (R₃₆ₐ)(R₃₇ₐ)(R₃₈ₐ)N—(CH₂)₃ group, R₃₆ₐ, R₃₇ₐ, R₃₈ₐ, R₃₈, R₃₉, R₄₀ and R₄₁, which may be identical or different, are chosen from hydrogen or an alkyl group comprising from 1 to 4 carbon atoms, and

X⁻ is an anion, in particular chosen from the group of halides, acetates, phosphates, nitrates and methyl sulfates.

Such compounds are, for example, Finquat C T-P, sold by the company Finetex (Quaternium 89), and Finquat C T, sold by the company Finetex (Quaternium 75), (d) quaternary ammonium salts containing at least one ester function, such as those having formula (IV) below:

[chemical structure]

in which:

R₄₂ is chosen from C₁ to C₆ alkyl groups and C₁ to C₆ hydroxyalkyl or dihydroxyalkyl groups;

R₄₃ is chosen from:

[chemical structure]

the group
groups R₄₇, which are linear or branched, saturated or unsaturated C₁ to C₂₂ hydrocarbon-based groups,
a hydrogen atom,

R₄₅ is chosen from:

[chemical structure]

the group
groups R₄₉, which are linear or branched, saturated or unsaturated C₁ to C₆ hydrocarbon-based groups,
a hydrogen atom,

R₄₄, R₄₆ and R₄₈, which may be identical or different, are chosen from linear or branched, saturated or unsaturated C₇ to C₂₁ hydrocarbon-based groups;

r, s and t, which may be identical or different, are integers ranging from 2 to 6;

y is an integer ranging from 1 to 10;

x and z, which may be identical or different, are integers ranging from 0 to 10;

X⁻ is a simple or complex and organic or mineral anion;

with the proviso that the sum x+y+z is from 1 to 15, that when x is 0 then R₄₃ denotes R₄₇, and that when z is 0 then R₄₅ denotes R₄₉.

The alkyl groups R₄₂ may be linear or branched, and more particularly linear.

Preferably, R₄₂ denotes a methyl, ethyl, hydroxyethyl or dihydroxypropyl group, and more particularly a methyl or ethyl group.

Advantageously, the sum x+y+z is from 1 to 10.

When R₄₃ is a hydrocarbon-based group R₄₉, it may comprise from 12 to 22 carbon atoms, or else from 1 to 3 carbon atoms.

When R₄₅ is a hydrocarbon-based group R₄₉, it preferably contains 1 to 3 carbon atoms.

Advantageously, R₄₄, R₄₆ and R₄₈, which may be identical or different, are chosen from linear or branched, saturated or unsaturated C₁₁ to C₂₁ hydrocarbon-based groups, and more particularly from linear or branched, saturated or unsaturated C₁₁ to C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion that is compatible with the ammonium bearing an ester function.

The anion X⁻ is even more particularly chloride or methyl sulfate.

Use is more particularly made, in the composition according to the invention, of the ammonium salts of formula (IV) in which:

R₄₂ denotes a methyl or ethyl group,

x and y are equal to 1;

z is equal to 0 or 1;

r, s and t are equal to 2;

R₄₃ is chosen from:

[chemical structure]

the group
methyl, ethyl or C₁₄ to C₂₂ hydrocarbon-based groups,
a hydrogen atom;

R₄₅ is chosen from:

[chemical structure]

the group
a hydrogen atom;

R₄₄, R₄₆ and R₄₈, which may be identical or different, are chosen from saturated or unsaturated, linear or branched C₁₃ to C₁₇ hydrocarbon-based groups and preferably from saturated or unsaturated, linear or branched C₁₃ to C₁₇ alkyl and alkenyl groups.

Advantageously, the hydrocarbon-based groups are linear.

Mention may for example be made of the salts (chloride or methyl sulfate in particular) of diacyloxyethyldimethylammonium, of diacyloxyethylhydroxyethyldimethylammonium, of monoacyloxyethylhydroxyethyldimethylammonium, of triacyloxyethylmethylammonium, of monoacyloxyethylhydroxyethyldimethylammonium, and mixtures thereof. The acyl groups preferably contain 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with C₁₀ to C₃₀ fatty acids or with mixtures of C₁₀ to C₃₀ fatty acids of plant or animal origin, or by transesterification of their methyl esters. This esterification is followed by a quaternization using an alkylating agent such as an alkyl halide (preferably a methyl or ethyl halide), a dialkyl sulfate (preferably a methyl or ethyl sulfate), methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

Preferably, the composition according the invention comprises one or more cationic surfactants of formula (Ib), and more particularly one or more cationic surfactants chosen from (C8-C30)alkyltrimethylammonium salts, and in particular cetyltrimethylammonium salts, behenyltrimethylammonium salts, palmitylamidopropyltrimethylammonium salts, and mixtures thereof.

More particularly, the composition comprises one or more cationic surfactants chosen from (C8-C30)alkyltrimethylammonium halides, in particular chlorides, cetyltrimethylammonium halides, in particular chloride, behenyltrimethylammonium halides, in particular chloride, palmitylamidopropyltrimethylammonium halides, in particular chloride; or (C8-C30)alkyltrimethylammonium (C1-C4)alkyl sulfates, in particular methosulfates, and in particular cetyltrimethylammonium (C1-C4)alkyl sulfates, in particular methosulfate, behenyltrimethylammonium (C1-C4)alkyl sulfates, in particular methosulfate, palmitylamidopropyltrimethylammonium (C1-C4)alkyl sulfates, in particular methosulfate; and also mixtures thereof.

Preferentially, the composition according to the invention comprises one or more cationic surfactants chosen from cetyltrimethylammonium halides, in particular chlorides, or behenyltrimethylammonium halides, in particular chlorides, and cetyltrimethylammonium (C1-C4)alkyl sulfates, in particular methosulfates, or behenyltrimethylammonium (C1-C4)alkyl sulfates, in particular methosulfates.

The composition according to the invention preferably comprises the surfactants different from the cationic surfactants of formula (Ia) in a total amount ranging from 0.1% to 10% by weight, better still from 0.2% to 10% by weight, preferentially from 0.5% to 5% by weight, relative to the total weight of the composition.

Preferentially, the composition according to the invention preferably comprises the surfactant(s) of formula (Ib), when they are present, in a total amount ranging from 0.1% to 10% by weight, better still from 0.2% to 10% by weight, preferentially from 0.5% to 5% by weight, relative to the total weight of the composition.

3/ Silicone Emulsions

The composition according to the invention also comprises an oil-in-water emulsion having a particle size D50 of less than 350 nm, and comprising:

a silicone mixture comprising (i) a polydialkylsiloxane comprising trialkylsilyl end groups, having a viscosity at 25° C. ranging from 40 000 to 100 000 mPa·s and (ii) an aminosilicone having a viscosity at 25° C. ranging from 1000 to 15 000 mPa·s and an amine number ranging from 2 to 10 mg of KOH per gram of aminosilicone;

a surfactant mixture comprising one or more non-ionic surfactants, said mixture having an HLB ranging from 10 to 16, and

water.

In the oil-in-water, or silicone-in-water, emulsion, according to the invention, a liquid phase (the dispersed phase) is advantageously dispersed in another liquid phase (the continuous phase); in the present invention, the mixture of silicones, or silicone phase, is dispersed in the aqueous continuous phase.

The mixture of silicones (or silicone mixture) comprises one or more polydialkylsiloxanes comprising trialkylsilyl end groups, preferably of formula (I):

R′3SiO(R′2SiO)pSiR′3

in which:

R′, which may be identical or different, is a monovalent hydrocarbon-based radical having from 1 to 18 carbon atoms, preferably from 1 to 6 carbon atoms, better still from 1 to 3 carbon atoms, even better still a methyl radical, and

p is an integer ranging from 500 to 2000, better still from 1000 to 2000.

The polydialkylsiloxanes comprising trialkylsilyl end groups according to the invention have a viscosity ranging from 40 000 to 100 000 mPa·s (preferably 100 000 excluded) at 25° C., preferably ranging from 40 000 to 70 000 mPa·s at 25° C., better still from 51 000 to 70 000 mPa·s at 25° C.

The polydialkylsiloxanes comprising trialkylsilyl end groups according to the invention are preferably linear, but they may comprise, in addition to the R′2SiO_{2/2} units (D-units), additional RSiO_{3/2} units (T-units) and/or SiO_{4/2} units (Q-units), in which R′, which may be identical or different, is a C1-018 monovalent hydrocarbon-based radical.

In formula (I), R′, which may be identical or different, is preferably:

an alkyl, preferably C1-C28 alkyl, radical, such as the radicals methyl, ethyl, n-propyl, isopropyl, 1-n-butyl, 2-n-butyl, isobutyl, tert-butyl, n-pentyl, isopentyl, neopentyl, tert-pentyl, hexyl and in particular n-hexyl, heptyl and in particular n-heptyl, octyl and in particular n-octyl, isooctyl, 2,2,4-trimethylpentyl; nonyl and in particular n-nonyl; decyl and in particular n-decyl; dodecyl and in particular n-dodecyl; octadecyl and in particular n-octadecyl;

an alkenyl radical such as vinyl and allyl;

a cycloalkyl radical such as cyclopentyl, cyclohexyl, cycloheptyl and methylcyclohexyl;

an aryl radical such as phenyl, naphthyl, anthryl and phenanthryl;

an alkaryl radical such as the radicals o-, m- and p-tolyl; xylyl, ethylphenyl;

an aralkyl radical such as benzyl and phenylethyl.

Preferentially, R′ is a methyl radical.

Preferably, the polydialkylsiloxanes comprising trialkylsilyl end groups are polydimethylsiloxanes (PDMSs) comprising trialkylsilyl end groups.

The silicone mixture also comprises one or more aminosilicones, preferably of formula (II): XR₂Si(OSiAR)ₙ(OSiR₂)ₘOSiR₂X

in which:

R, which may be identical or different, is a monovalent hydrocarbon-based radical having from 1 to 18 carbon atoms, preferably from 1 to 6 carbon atoms, better still from 1 to 3 carbon atoms, even better still a methyl radical,

X, which may be identical or different, represents R or a hydroxyl (OH) ora C1-C6 alkoxy group; preferably X is R, that is to say a monovalent hydrocarbon-based radical having from 1 to 18 carbon atoms, preferably from 1 to 6 carbon atoms, better still from 1 to 3 carbon atoms, even better still a methyl radical,

A is an amino radical of formula —R¹—[NR²—R³—]ₓNR²₂, or the protonated form of this amino radical, with

R¹ representing a C1-C6 alkylene radical, preferably a —CH₂CH₂CH₂— or

CH₂CH(CH₃)CH₂— radical,

R², which may be identical or different, being a hydrogen atom ora C1-C4 alkyl radical, preferably a hydrogen atom,

R³ being a C1-C6 alkylene radical, preferably a —CH₂CH₂— radical,

x being 0 or 1;

m and n are integers such that m+n ranges from 50 to 1000, better still from 50 to 600.

Preferably, A is an amino radical of formula —R¹—[NR²—R³—]ₓNR²₂, or the protonated form of this amino radical, with R¹ being —CH₂CH₂CH₂— or —CH₂CH(CH₃)CH₂—, R² being hydrogen atoms, R³ being —CH₂CH₂— and x being equal to 1.

Preferably, R, which may be identical or different, is:

an alkyl, preferably C1-028 alkyl, radical, such as the radicals methyl, ethyl, n-propyl, isopropyl, 1-n-butyl, 2-n-butyl, isobutyl, tert-butyl, n-pentyl, isopentyl, neopentyl, tert-pentyl, hexyl and in particular n-hexyl, heptyl and in particular n-heptyl, octyl and in particular n-octyl, isooctyl, 2,2,4-trimethylpentyl; nonyl and in particular n-nonyl; decyl and in particular n-decyl; dodecyl and in particular n-dodecyl; octadecyl and in particular n-octadecyl;

an alkenyl radical such as vinyl and allyl;

a cycloalkyl radical such as cyclopentyl, cyclohexyl, cycloheptyl and methylcyclohexyl;

an aryl radical such as phenyl, naphthyl, anthryl and phenanthryl;

an alkaryl radical such as the radicals o-, m- and p-tolyl; xylyl, ethylphenyl;

an aralkyl radical such as benzyl and phenylethyl.

Preferentially, R is a methyl radical.

The aminosilicones according to the invention have a viscosity, measured at 25° C., ranging from 1000 to 15 000 mPa·s, preferably ranging from 1500 to 15000 mPa·s.

The aminosilicones according to the invention have an amine number ranging from 2 to 10 mg of KOH per gram of aminosilicone; preferably from 3.5 to 8 mg.

The molar percentage of amine function is preferably between 0.3 and 8 mol %.

As examples of aminosilicones, mention may be made of aminosilicones comprising trialkylsilyl end groups; preferably aminoethylaminopropylmethylsiloxanes comprising trialkylsilyl end groups, even better still copolymers of aminoethylaminopropylmethylsiloxane comprising trialkylsilyl end groups/dimethylsiloxane.

The amino radical A may be partially or totally protonated, for example by addition of acids to the aminosilicone, so as to obtain the salified form of said amino radical.

As acids that may be used, mention may be made of linear or branched carboxylic acids having from 3 to 18 carbon atoms, such as formic acid, acetic acid, propionic acid, butyric acid, pivalic acid, sorbic acid, benzoic acid or salicylic acid. Preferably, the acids may be used in a proportion of from 0.1 to 2.0 mol per mole of amino radical A in the aminosilicone of formula (II).

The silicone mixture preferably comprises (i) one or more polydialkylsiloxanes comprising trialkylsilyl end groups, having a viscosity, at 25° C., ranging from 40 000 to 100 000 mPa·s, in an amount of from 70% to 90% by weight, preferably from 75% to 85% by weight, and (ii) one or more aminosilicones having a viscosity, at 25° C., ranging from 1000 to 15 000 mPa·s and an amine number ranging from 2 to 10 mg of KOH per gram of aminosilicone, in an amount of from 10% to 30% by weight, in particular from 15% to 25% by weight, relative to the total weight of the silicone mixture.

The oil-in-water emulsion also comprises a surfactant mixture which comprises one or more non-ionic surfactants; said surfactant mixture may optionally comprise one or more cationic surfactants.

Said surfactant mixture has an HLB ranging from 10 to 16.

The non-ionic surfactants that may be used may be chosen from alcohols, α-diols and (C1-20)alkylphenols, these compounds being polyethoxylated and/or polypropoxylated and/or polyglycerolated, the number of ethylene oxide and/or propylene oxide groups possibly ranging from 1 to 100, and the number of glycerol groups possibly ranging from 2 to 30; or else these compounds comprising at least one fatty chain comprising from 8 to 30 carbon atoms and especially from 16 to 30 carbon atoms.

Mention may also be made of condensates of ethylene oxide and of propylene oxide with fatty alcohols; polyethoxylated fatty amides preferably having from 2 to 30 ethylene oxide units, polyglycerolated fatty amides comprising on average from 1 to 5, and in particular from 1.5 to 4, glycerol groups; ethoxylated fatty acid esters of sorbitan preferably containing from 2 to 40 ethylene oxide units, fatty acid esters of sucrose, polyoxyalkylenated and preferably polyoxyethylenated fatty acid esters containing from 2 to 150 mol of ethylene oxide, including oxyethylenated plant oils, N—(C6-C24 alkyl)glucamine derivatives, amine oxides such as (C10-C14 alkyl)amine oxides or N—(C10-C14 acyl)aminopropylmorpholine oxides.

Mention may also be made of non-ionic surfactants of alkyl(poly)glycoside type, represented especially by the following general formula: R₁O—(R₂O)ₜ—(G)ᵥ

in which:

R₁ represents a linear or branched alkyl or alkenyl radical comprising 6 to 24 carbon atoms and especially 8 to 18 carbon atoms, or an alkylphenyl radical of which the linear or branched alkyl radical comprises 6 to 24 carbon atoms and especially 8 to 18 carbon atoms;

R₂ represents an alkylene radical comprising 2 to 4 carbon atoms,

G represents a sugar unit comprising 5 to 6 carbon atoms,

t denotes a value ranging from 0 to 10 and preferably 0 to 4,

v denotes a value ranging from 1 to 15 and preferably 1 to 4.

Preferably, the alkyl(poly)glycoside surfactants are compounds of the formula described above in which:

R₁ denotes a linear or branched, saturated or unsaturated alkyl radical comprising from 8 to 18 carbon atoms,

R₂ represents an alkylene radical comprising 2 to 4 carbon atoms,

t denotes a value ranging from 0 to 3 and preferably equal to 0,

G denotes glucose, fructose or galactose, preferably glucose;

the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl(poly)glycoside surfactant is an alkyl(poly)glucoside surfactant. C8/C16 alkyl(poly)glucosides 1,4, and in particular decyl glucosides and caprylyl/capryl glucosides, are most particularly preferred.

Among commercial products, mention may be made of the products sold by the company Cognis under the names Plantaren® (600 CS/U, 1200 and 2000) or Plantacare® (818, 1200 and 2000); the products sold by the company SEPPIC under the names Oramix CG 110 and Oramix® NS 10; the products sold by the company BASF under the name Lutensol GD 70, or else the products sold by the company Chem Y under the name AG10 LK.

Preferably, use is made of C8/C16-alkyl (poly)glycosides 1,4, in particular as an aqueous 53% solution, such as those sold by Cognis under the reference Plantacare® 818 UP.

The mono- or polyglycerolated surfactants preferably comprise an average number of glycerol groups ranging from 1 to 30, in particular from 1 to 10, better still from 1.5 to 5. They preferably correspond to one of the following formulae:

RO[CH2CH(CH2OH)O]ₘH,

RO[CH2CH(OH)CH2O]ₘH or

RO[CH(CH2OH)CH2O]ₘH;

in which:

R represents a saturated or unsaturated, linear or branched hydrocarbon-based (in particular alkyl or alkenyl) radical comprising 8 to 40 carbon atoms, in particular 10 to 30 carbon atoms, optionally comprising one or more heteroatoms such as O and N; and

m is an integer ranging from 1 to 30, preferably from 1 to 10, better still from 1.5 to 6.

In particular, R may comprise one or more hydroxyl and/or ether and/or amide groups. Preferably, R is a mono- or polyhydroxylated C10-C20 alkyl or alkenyl radical.

Mention may be made of polyglycerolated (3.5 mol) hydroxylauryl ether, such as the product Chimexane® NF from Chimex.

Mention may also be made of (poly)ethoxylated fatty alcohols preferably comprising one or more saturated or unsaturated, linear or branched hydrocarbon-based chains comprising 8 to 30 carbon atoms, preferably from 12 to 22 carbon atoms, optionally substituted with one or more hydroxyl (OH) groups, in particular 1 to 4 hydroxyl groups.

When the chain is unsaturated, it may comprise one to three conjugated or non-conjugated carbon-carbon double bonds.

The (poly)ethoxylated fatty alcohols preferably correspond to formula (II):

R3—(OCH2CH2)_{c}OH

in which:

R3 represents a linear or branched alkyl or alkenyl radical comprising 8 to 40 carbon atoms and in particular 8 to 30 carbon atoms, optionally substituted with one or more, in particular 1 to 4, hydroxyl groups; and

c is an integer ranging from 1 to 200, preferably from 2 to 150, or even from 4 to 50 and even better still from 8 to 30.

The (poly)ethoxylated fatty alcohols are more particularly fatty alcohols comprising 8 to 22 carbon atoms, oxyethylenated with 1 to 30 mol of ethylene oxide (1 to 30 EO); mention may in particular be made of lauryl alcohol 2 EO; lauryl alcohol 3 EO; decyl alcohol 3 EO; decyl alcohol 5 EO and oley alcohol 20 EO.

The non-ionic surfactants may advantageously be chosen from:

(i) (poly)oxyalkylenated, in particular (poly)ethoxylated, fatty alcohols, and in particular those of formula: R₃—(OCH₂CH₂)_{c}OH in which:

R3 represents a linear or branched alkyl or alkenyl radical comprising 8 to 40 carbon atoms and in particular 8 to 30 carbon atoms, optionally substituted with one or more, in particular 1 to 4, hydroxyl groups; and

c is an integer ranging from 1 to 200, in particular from 2 to 150, or even from 4 to 50 and even better still from 8 to 20;

(ii) (poly)oxyalkylenated (C8-C32)alkyl phenyl ethers, in particular comprising from 1 to 200, better still from 1 to 30 mol of ethylene oxide;

(iii) polyoxyalkylenated esters of C8-C32 fatty acids and of sorbitan, in particular polyoxyethylenated esters of C8-C32 fatty acids and of sorbitan, preferably having from 2 to 40 ethylene oxide units, better still from 2 to 20 ethylene oxide (EO) units; in particular polyoxyethylenated esters of C10-C24 fatty acids and of sorbitan, preferably having from 2 to 40 ethylene oxide units, better still from 2 to 20 ethylene oxide (EO) units; and

(iv) polyoxyethylenated esters of C8-C32 fatty acids, preferably having from 2 to 150 ethylene oxide units; in particular polyoxyethylenated esters of C10-C24 fatty acids, comprising in particular 2 to 150 ethylene oxide (EO) units.

The non-ionic surfactants may advantageously be chosen from alkyl ethers and alkyl esters of polyalkylene glycol, in particular of polyethylene glycol.

Mention may in particular be made of:

polyethylene glycol octyl ether; polyethylene glycol lauryl ether; polyethylene glycol tridecyl ether; polyethylene glycol cetyl ether; polyethylene glycol stearyl ether; and most particularly trideceth-3, trideceth-10 and steareth-6;

polyethylene glycol nonylphenyl ether; polyethylene glycol dodecylphenyl ether; polyethylene glycol cetylphenyl ether; polyethylene glycol stearylphenyl ether;

polyethylene glycol sorbitan monostearate, polyethylene glycol sorbitan monooleate;

polyethylene glycol stearate, and in particular PEG100 stearate.

Even better still, the non-ionic surfactants may be chosen from Steareth-6, PEG100 stearate, trideceth-3 and trideceth-10, and mixtures thereof; most particularly, a mixture comprising these four non-ionic surfactants.

The surfactant mixture may optionally comprise one or more cationic surfactants, which may be chosen from tetraalkylammonium, tetraarylammonium and tetraalkylarylammonium salts, in particular halides, and most particularly from cetrimonium or behentrimonium salts, in particular halides, better still chlorides.

The oil-in-water emulsion preferably comprises the surfactant mixture in a total amount ranging from 5% to 15% by weight, in particular from 8% to 15% by weight, even better still from 10% to 12% by weight, relative to the total weight of the emulsion.

The oil-in-water emulsion preferably comprises the non-ionic surfactant(s) in a total amount ranging from 5% to 15% by weight, in particular from 8% to 15% by weight, even better still from 10% to 12% by weight, relative to the total weight of the emulsion.

The oil-in-water emulsion preferably comprises the cationic surfactant(s), when they are present, in a total amount ranging from 0.5% to 1.5% by weight relative to the total weight of the emulsion.

The oil-in-water emulsion preferably comprises the silicone mixture in a total amount ranging from 40% to 60% by weight, in particular from 45% to 55% by weight, relative to the total weight of the emulsion.

The oil-in-water emulsion preferably comprises the polydialkylsiloxane(s) comprising trialkylsilyl end groups in a total amount ranging from 35% to 45% by weight, in particular from 38% to 42% by weight, relative to the total weight of the emulsion.

The oil-in-water emulsion preferably comprises the aminosilicone(s) in a total amount ranging from 5% to 15% by weight, in particular from 8% to 12% by weight, relative to the total weight of the emulsion.

The oil-in-water emulsion preferably comprises water in a total amount ranging from 25% to 50% by weight, in particular from 30% to 45% by weight, even better still from 35% to 42% by weight, relative to the total weight of the emulsion.

The oil-in-water emulsion may also comprise a preservative, such as phenoxyethanol, in an amount ranging from 0.5% to 1% by weight relative to the total weight of the emulsion.

A process for preparing the oil-in-water emulsion preferably comprises:

a step of mixing one or more polydialkylsiloxanes comprising trialkylsilyl end groups, having a viscosity, at 25° C., ranging from 40 000 to 100 000 mPa·s, and one or more aminosilicones having a viscosity, at 25° C., ranging from 1000 to 15 000 mPa·s and an amine number ranging from 2 to 10 mg of KOH per gram of aminosilicone; at a temperature of from 15° C. to 40° C., in particular at 25° C., in order to obtain a fluid mixture of silicones; then

a step of adding a surfactant mixture comprising one or more non-ionic surfactants, said mixture having an HLB ranging from 10 to 16, to said fluid mixture of silicones, in order to obtain an emulsified silicone mixture; then

a step of homogenizing said emulsified silicone mixture, followed by

a step of adding water, in particular demineralized water, preferentially in steps, in order to obtain an oil-in-water emulsion having a particle size D50 of less than 350 nm.

The preparation process may also comprise an additional step of adding one or more preservatives.

The pH of the oil-in-water emulsion is generally between 4 and 6.

The oil-in-water emulsion has a particle size D50 of less than 350 nm, in particular of between 100 and 300 nm, better still between 150 and 250 nm, or even between 160 and 200 nm.

This size corresponds to the average hydrodynamic particle diameter. The particle size D50 is expressed by volume. It can be measured using a ZetaSizer device from Malvern, UK, model Nano-ZS, based on the “Photon Correlation Spectroscopy (PCS)” method.

Method for Measuring the Particle Size

The particle size of the emulsion is measured using a ZetaSizer device from Malvern, UK, model Nano-ZS, based on the “Photon Correlation Spectroscopy (PCS)” method.

The particle size D50 is measured when the evaluation algorithm is “cumulant analysis”.

0.5 g of the emulsion is placed in a 250 ml beaker, 100 ml of demineralized water are added and mixing is carried out in order to obtain the solution to be tested. The solution to be tested is placed in the measuring vessel (or cell) and introduced into the measuring device.

The size D₅₀ corresponds to the particle diameter value at 50% in cumulative distribution.

For example, if D_{50b =170} nm, this means that 50% of the particles have a size of greater than 170 nm, and that 50% of the particles have a size of less than 170 nm.

It should be recalled that this distribution is by volume.

Method for Measuring the Viscosity

The viscosities, in particular of the silicone compounds, are measured at 25° C., 1 atm. To measure viscosities of between 1000 and 40 000 mPa·s at 25° C., use may be made of an Anton Paar rheometer, model MCR101, cylinder geometry, single gap: CC27 spindle, shear rate1 s⁻¹ for 2 minutes, at 25° C.

To measure viscosities of between 40 000 and 100 000 mPa·s at 25° C., use may be made of an Anton Paar rheometer, model MCR101, 25-6 cone (cone-plate geometry, 25 mm in diameter/6° cone); Zero gap, shear rate 1 s⁻¹ for 2 minutes, at 25° C.

Three measurements are carried out for each sample, and the viscosity value is taken at 60 seconds. The MCR Rheometer Series products operate according to the USP convention (US Pharmacopeia Convention, 912—Rotational Rheometer methods).

Method for Measuring the Amine Number

The amine number can be measured by acid-based titration, using a potentiometer [Make: Veego; model VPT-MG].

0.6 g of sample is placed in a 500 ml beaker and a 1:1 toluene-butanol mixture is added, then mixing is carried out.

The solution is titrated with a 0.1 N HCI solution. A determination of the zero value (V_{blank}) is also carried out with the 1:1 toluene-butanol mixture alone.

The amine number is calculated by means of the formula:

56.11×(V−V_{Blank})×N/ W mg KOH/g of sample

With V=volume of HCl required (in ml), V_{Blank}=volume of HCl required for the zero value (in ml); N=normality of HCl, i.e. 0.1, and W=weight of the sample (in g).

HLB Values

The term HLB relates to the hydrophobicity-lipophilicity balance of a surfactant. It can be measured experimentally or calculated.

In the present application, the HLB values are the values at 25° C.

The HLB values can be calculated by means of the following equation: HLB=(E +P)/5, in which E is the % by weight of oxyethylene and P is the % by weight of polyol, as is described in the publication Griffin, J. Soc. Cosm. Chem. 1954 (vol.5, n° 4), pages 249-256.

The HLB values can also be determined experimentally according to the book by Puisieux and Seiller, entitled “Galenica 5: Les systemes disperses [Galenics 5: Dispersed systems]—Volume I—Agents de surface et emulsions [Surface agents and emulsions]—Chapter IV—Notions de HLB et de HLB critique [Notions of HLB and of critical HLB], pages 153-194—paragraph 1.1.2. Determination de HLB par voie experimentale [Experimental determination of HLB], pages 164-180”.

Preferably, the HLB values that will be taken into account are those obtained by calculation, in particular in the following way: “calculated HLB”=20×(molar mass of the hydrophilic part/total molar mass).

Thus, for an oxyethylenated fatty alcohol, the hydrophilic part corresponds to the oxyethylene units fused to the fatty alcohol and the “calculated HLB” then corresponds to the “HLB according to Griffin”.

For an ester or an amide, the hydrophilic part is generally defined as being beyond the carbonyl group, starting from the fatty chain(s).

The HLB values of non-ionic surfactants can also be calculated by means of the Davies formula, as described in Davies J T (1957), “A quantitative kinetic theory of emulsion type, I. Physical chemistry of the emulsifying agent”, Gas/Liquid and Liquid/Liquid Interface (Proceedings of the International Congress of Surface Activity): 426-438.

According to this formula, the HLB value is obtained by adding the hydrophilic/hydrophobic contribution the to the constituent groups in the surfactant:

HLB=(number of hydrophilic groups)−n(number of groups per CH₂ group)+7.

The HLB values of some cationic surfactants are given in Table IV, in “Cationic emulsifiers in cosmetics”, GODFREY, J. Soc. Cosmetic Chemists (1966) 17, pages 17-27.

When two surfactants A and B, of known HLB values, are mixed, the HLB_{Mix} corresponds to the HLB of the mixture and can be expressed by the following equation:

HLB_{Mix}=(W_{A}HLB_{A}+W_{B}HLB_{B})/(W_{A}+W_{B})

in which W_{A} is the amount (weight) of the 1^{st} surfactant A and W_{B} the amount of the 2^{nd} surfactant B, and HLB_{A} and HLB_{B} are the HLB values of the surfactant A and of the surfactant B.

Preferably, the composition according to the invention comprises the oil-in-water emulsion in a total amount ranging from 0.1% to 10% by weight, better still from 0.2% to 8% by weight, preferentially from 0.5% to 6% by weight, relative to the total weight of the composition.

Preferably, the composition according to the invention comprises the oil-in-water emulsion in a total amount ranging from 0.1% to 10% by weight, better still from 0.2% to 8% by weight, preferentially from 0.5% to 6% by weight, relative to the total weight of the composition, and the emulsion has a solids (or active material) content of between 40% and 60% by weight, in particular 45% to 55% by weight, relative to the total weight of the emulsion.

4/Fatty Alcohols

The composition according to the invention may also comprise one or more fatty alcohols.

For the purposes of the present invention, the term fatty alcohol is intended to mean an alcohol comprising, in its main chain, at least one saturated or unsaturated, linear or branched hydrocarbon-based chain, such as alkyl or alkenyl, comprising at least 8 carbon atoms, preferably from 8 to 30 carbon atoms, and even better still from 10 to 22 carbon atoms. They are not glycerolated or polyoxyalkylenated.

They preferably have the structure R—OH in which R denotes a linear or branched, saturated or unsaturated C8-C30, preferably C10-C22, better still C12-C24 alkyl or alkenyl group, R possibly being substituted by one or more hydroxyl groups.

The fatty alcohols may be liquid or solid at ambient temperature (25° C.) and at atmospheric pressure (760 mmHg, i.e. 1.013×10⁵ Pa).

The liquid fatty alcohols of the invention may be saturated or unsaturated.

The saturated liquid fatty alcohols are preferably branched. They may optionally comprise in their structure at least one aromatic or non-aromatic ring. Preferably, they are acyclic.

The unsaturated liquid fatty alcohols contain in their structure at least one double or triple bond, and preferably one or more double bonds. When several double bonds are present, there are preferably 2 or 3 of them, and they may be conjugated or unconjugated. These unsaturated liquid fatty alcohols may be linear or branched. They may optionally comprise in their structure at least one aromatic or non-aromatic ring. Preferably, they are acyclic.

The liquid fatty alcohols preferably have the structure R′—OH, in which R′ denotes a branched C12-C24 alkyl or linear or branched C12-C24 alkenyl group, R′ possibly being substituted by one or more hydroxyl groups. Preferably, R′ is a branched C12-C24 alkyl group, optionally substituted with one or more hydroxyl groups; better still, R′ does not contain a hydroxyl group.

The solid fatty alcohols are more particularly chosen from linear or branched, saturated or unsaturated alcohols comprising from 8 to 30 carbon atoms and preferably from 8 to 24 carbon atoms.

Preferably, the solid fatty alcohols have the structure R″-OH with R″ denoting a linear alkyl group, optionally substituted by one or more hydroxyl groups, comprising from 8 to 30, better still from 10 to 30, or even from 12 to 24 carbon atoms.

They are preferably chosen from saturated or unsaturated, linear or branched, preferably linear and saturated, (mono)alcohols comprising from 8 to 30 carbon atoms, better still from 10 to 30, or even from 12 to 24 carbon atoms.

Preferably, the composition comprises one or more solid fatty alcohols.

As examples of fatty alcohols that may be used in the present invention, mention may be made of oleyl alcohol, linoleyl alcohol, linolenyl alcohol, undecylenyl alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol, cetyl alcohol, stearyl alcohol, cetylstearyl alcohol, and mixtures thereof.

Preferentially, 2-octyl-1-dodecanol, oleyl alcohol, cetyl alcohol, stearyl alcohol, cetylstearyl alcohol, and mixtures thereof, may be used.

The composition according to the invention can comprise the fatty alcohol(s) in a total amount ranging from 0.1% to 20% by weight, better still from 0.5% to 15% by weight and preferentially from 1% to 10% by weight, relative to the total weight of the composition.

5/ Other Ingredients

The composition according to the invention can also comprise one or more additional fatty substances, different from the silicones present in the silicone emulsion above, and from the fatty alcohols above, preferably one or more non-silicone fatty substances which are liquid or solid at 25° C., 1 atm., in particular chosen from liquid or solid fatty esters.

The term “fatty ester” is intended to mean an ester comprising one or more linear or branched, saturated or unsaturated hydrocarbon-based chains comprising from 6 to 30 carbon atoms, optionally substituted in particular with one or more hydroxyl groups (in particular 1 to 4). If they are unsaturated, these compounds may comprise one to three conjugated or unconjugated carbon-carbon double bonds.

Mention may in particular be made of the esters of saturated or unsaturated, linear or branched C1-C26 aliphatic mono- or polyacids and of saturated or unsaturated, linear or branched C1-C26 aliphatic mono- or polyalcohols, the total carbon number of the esters being more particularly greater than or equal to 10.

Preferably, for the esters of monoalcohols, at least one from among the alcohol and the acid from which the esters of the invention are derived is branched.

Among the monoesters of monoacids and of monoalcohols, mention may be made of alkyl, in particular C1-C28 alkyl, palmitates, such as ethyl palmitate or iso-propyl palmitate, alkyl, in particular C1-C28 alkyl, myristates such as isopropyl myristate or ethyl myristate, isocetyl stearate, 2-ethylhexyl isononanoate, isodecyl neopentanoate and isostearyl neopentanoate.

Esters of C4-C22 dicarboxylic or tricarboxylic acids and of C1-C22 alcohols and esters of mono-, di- or tricarboxylic acids and of di-, tri-, tetra- or pentahydroxy alcohols which are C2-C26 may also be used.

Mention may be made especially of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Mention may also be made of esters of sugars and of linear or branched, saturated or unsaturated C₆-C₃₀, preferably C₁₂-C₂₂, fatty acids

If they are unsaturated, these compounds may comprise one to three conjugated or unconjugated carbon-carbon double bonds.

The term “sugar” is intended to mean oxygen-bearing hydrocarbon-based compounds containing several alcohol functions, with or without aldehyde or ketone functions, and which comprise at least 4 carbon atoms. These sugars may be mon-osaccharides, oligosaccharides or polysaccharides. Preferably, these sugars are chosen from sucrose, glucose, galactose, ribose, fucose, maltose, fructose, mannose, arabinose, xylose and lactose, and derivatives thereof, especially alkyl derivatives, such as methyl derivatives, for instance methylglucose.

The esters according to this variant may be mono-, di-, tri- and tetraesters, or polyesters.

These esters may be, for example, oleates, laurates, palmitates, myristates, behenates, cocoates, stearates, linoleates, linolenates, caprates and arachidonates, and mixtures thereof, such as, especially, oleopalmitate, oleostearate or palmitostearate mixed esters.

More particularly, use is made of monoesters and diesters and especially of sucrose, glucose or methylglucose mono- or dioleates, stearates, behenates, oleopalmitates, linoleates, linolenates or oleostearates, or alternatively of methylglucose dioleate (Glucate® DO).

It is also possible to use pentaerythrityl esters, preferably pentaerythrityl tetraisostearate, pentaerythrityl tetraoctanoate, and caprylic and capric acid hexaesters as a mixture with dipentaerythritol.

Among the natural or synthetic monoacid, diacid or triacid esters of glycerol, mention may be made of plant oils or synthetic oils.

More particularly, said plant oils or synthetic oils are chosen from triglyceride oils of plant or synthetic origin, such as liquid fatty acid triglycerides containing from 6 to 30 carbon atoms, for instance heptanoic or octanoic acid triglycerides, or alternatively, for example, sesame oil, soybean oil, coffee oil, safflower oil, borage oil, sunflower oil, olive oil, apricot kernel oil, camellia oil, bambara pea oil, avocado oil, mango oil, rice bran oil, cottonseed oil, rose oil, kiwi seed oil, sea buckthorn pulp oil, blueberry seed oil, poppy seed oil, orange pip oil, sweet almond oil, palm oil, coconut oil, vernonia oil, marjoram oil, baobab oil, rapeseed oil, ximenia oil, pracaxi oil, caprylic/capric acid triglycerides such as those sold by the company Stearineries Dubois or those sold under the names Miglyol® 810, 812 and 818 by the company Dynamit Nobel, jojoba oil and shea butter oil.

Mention may preferably be made of triglycerides of plant origin, in particular oils chosen from avocado oil, olive oil, camellia oil and apricot kernel oil, and mixtures thereof, and C₄-C₂₂ dicarboxylic or tricarboxylic acid esters of C₁-C₂₂ alcohols, in particular 1,3-propanediol dicaprylate.

Preferentially, the esters may be chosen from ethyl, isopropyl, myristyl, cetyl or stearly palmitate, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The care composition may comprise the fatty substance(s), when they are present, in a content ranging from 0.01% to 20% by weight, preferably from 0.05% to 15% by weight and preferentially from 0.1% to 10% by weight, or even from 0.2% to 5% by weight, relative to the total weight of the composition.

The composition according to the invention is advantageously aqueous with a water content of preferably between 40% and 98% by weight, in particular between 50% and 98% by weight, or even between 60% and 95% by weight, preferentially between 70% and 95% by weight, relative to the total weight of the composition.

The pH of composition is preferably between 2.5 and 6, in particular between 2.5 and 5, or even between 2.8 and 4.

The composition according to the invention may also comprise one or more additives chosen from anionic, non-ionic, cationic or amphoteric polymers; ceramides, pseudo-ceramides, vitamins and provitamins including panthenol, water-soluble and liposoluble sunscreens, nacreous agents and opacifiers, sequestrants, solubilizers, antioxidants, antidandruff agents, anti-seborrhoeic agents, agents for preventing hair loss and/or for promoting hair regrowth, penetrants, fragrances, peptizers and preservatives. These additives may be present in the composition according to the invention in an amount ranging from 0 to 20% by weight, relative to the total weight of the composition. Those skilled in the art will take care to select these optional additives and the amounts thereof so that they do not harm the properties of the compositions of the present invention.

The compositions according to the invention may advantageously be in the form of a hair composition, in particular in the form of a rinse-off conditioner, of a mask or of a leave-on care product.

The present invention also relates to a cosmetic process for treating, more particularly for conditioning, keratin materials, and in particular keratin fibres, and in particular the hair, which comprises the application to said keratin materials of a composition as described above, optionally followed by a leave-on time and/or by a rinsing step and/or by a drying step.

The application may be performed on dry or wet hair, preferably wet hair.

The leave-on time of the composition on the keratin fibres may range from 5 seconds to 10 minutes, better still from 10 seconds to 5 minutes and even better still from 20 seconds to 2 minutes.

This application may or may not be followed by rinsing, and is preferably followed by rinsing.

The invention is illustrated in greater detail by the following examples. Unless otherwise indicated, the amounts indicated are expressed in % by weight of active material (AM) relative to the total weight of the composition.

### EXAMPLE 1

### Preparation of the Silicone Emulsion

450 g of fluid aminosilicone (copolymer of dimethylsiloxane—aminoethylaminopropylmethylsiloxane comprising trimethylsilyl end groups, having an amine number of 7.2 mg of KOH/g and a viscosity of 5600 mPa·s at 25° C.) are transferred into a 1st vessel; 1800 g of dimethylsiloxane comprising trimethylsilyl end groups, having a viscosity of 61 500 mPa·s at 25° C., are added, with stirring, and the stirring is maintained for 2 hours at ambient temperature.

In a separate vessel, 49 g of steareth-6 and 62 g of PEG100 stearate are mixed, and the mixture is heated to 60° C. The mixture is maintained at this temperature until a liquid mixture is obtained, then 31 g of trideceth-3 and 350 g of trideceth-10 (80% of active material) are added. The surfactant mixture has an HLB=11.25. 80 g of water and 6.2 g of glacial acetic acid are added and the stirring is continued until a creamy paste is obtained.

The content of this 2nd vessel (creamy paste) is then transferred into the 1st vessel (containing the silicones), then the mixture obtained is mixed for 30 minutes at ambient temperature (20-25° C.). The mixing steps are carried out in order to obtain a homogeneous mixture; they are carried out at ambient temperature.

79.6 g of demineralized water are added and mixing is carried out for 60 minutes.

72.7 g of demineralized water are added and mixing is carried out for 50 minutes.

197.4 g of demineralized water are added and mixing is carried out for 5 minutes.

294.3 g of demineralized water are added and mixing is carried out for 5 minutes.

180 g of demineralized water are added and mixing is carried out for 5 minutes.

180 g of demineralized water are added and mixing is carried out for 5 minutes.

197.4 g of demineralized water are added and mixing is carried out for 5 minutes. 197.4 g of demineralized water are added and mixing is carried out for 3 minutes.

228.5 g of demineralized water are added and mixing is carried out for 3 minutes.

Finally, 40.5 g of 2-phenoxyethanol (preservative) are added and mixing is carried out for 3 minutes.

An oil-in-water emulsion having a particle size D50 of 170 nm is obtained.

### EXAMPLE 2

The cosmetic compositions below comprising the following ingredients (amounts expressed as % by weight of active material AM, unless otherwise indicated) are prepared:

| Ingredients | Invention A | Comparative B | Comparative C |
|---|---|---|---|
| Cetearyl alcohol | 6% | 6% | 6% |
| Mixture of myristyl stearate and | 0.5% | 0.5% | 0.5% |
| myristyl palmitate (INCI: CETYL | | | |
| ESTERS) | | | |
| Silicone emulsion as prepared | 2% (0.8% AM | 2% (0.8% AM | 2% (0.8% AM |
| in Example 1 | of silicone + | of silicone + | of silicone + |
| | 0.2% AM of | 0.2% AM of | 0.2% AM of |
| | aminosilicone) | aminosilicone) | aminosilicone) |
| Dicetyldimethylammonium | 1% AM | 2% AM | — |
| chloride | | | |
| (VARISOFT 432 CG from | | | |
| Evonik) | | | |
| Cetyltrimethylammonium | 1% AM | — | 2% AM |
| chloride | | | |
| (DEHYQUART A OR from | | | |
| BASF) | | | |
| Preservative, fragrance | qs | qs | qs |
| Water | qs 100% | qs 100% | qs 100% |

Compositions are obtained which can be used as rinse-off conditioner.

a) Viscosity

The viscosity of the 3 compositions was measured 24 h after production, then after two months of storage at ambient temperature (25° C.) (Rheomat RM 200, 25° C., spindle 3, 200 rpm).

The results obtained are given below:

| | Viscosity 24 h | Viscosity after | |
|---|---|---|---|
| | after production | 2 months at 25° C. | % variation |
| A (invention) | 1145 cps | 1270 cps | +10.9% |
| B (comparative) | 815 cps | 650 cps | −20.2% |
| C (comparative) | 550 cps | 690 cps | +25.5% |

The composition A according to the invention has a higher viscosity than the comparative compositions B and C, which allows easy application without the risk of running.

After a storage period of 2 months at 25° C., the change in the viscosity, compared with the initial viscosity, is smaller for the composition A according to the invention compared with the compositions B and C.

b) Deposit of Fatty Substances and of Cations

The compositions A, B and C were applied to pads of hair trimmings weighing 200 mg, previously washed with a standard shampoo and rinsed, in a proportion of 1 ml of composition per pad of hair.

After rinsing and drying, the deposit of, on the one hand, fatty substances and of, on the other hand, cations was measured by gas chromatography after desorption in dichloromethane.

The following results are obtained (in micrograms/gram of hair)

| | Total fatty substances | Total cations |
|---|---|---|
| | (μg/g hair) | (μg/g hair) |
| A (invention) | 2925 | 755 |
| B (comparative) | 7143 | 713 |
| C (comparative) | 2328 | 1197 |

It is noted that the composition A makes it possible to deposit amounts of fatty substances and of cations that are intermediate between the composition B and the composition C.

Excessively large deposits of fatty substances result in an unclean greasy feel, contrary to the desired natural and clean feel.

Excessively large deposits of cations result in a softening of the fibres, contrary to the desired tonicity effect.

These results are confirmed by the rubbing and bending force measurements, carried out on locks, below.

c) Rubbing

Locks of hair, washed beforehand with a standard shampoo and rinsed, are treated with each of the compositions A, B and C, in a proportion of 0.4 g of composition per g of hair.

After rinsing and drying, 4 lock portions, each weighing 150 milligrams, are sampled from each lock, and are each mounted on a support.

Each lock portion is then inserted between 2 jaws: the maximum force required to move the support along the 2 jaws is then recorded.

The mean force corresponding to the mean of the 4 maximum forces measured is calculated.

The lower the rubbing force, the more representative is the surface of the lock of an unclean greasy feel.

The following results are obtained:

| | Mean rubbing force (in N) |
|---|---|
| A (invention) | 0.334 |
| B (comparative) | 0.143 |
| C (comparative) | 0.147 |

The rubbing forces of the compositions B and C are lower than that of the composition A; this reflects a surface that is much more representative of an unclean greasy feel for the compositions B and C.

d) Bending

Locks of hair, washed beforehand with a standard shampoo and rinsed, are treated with each of the compositions A, B and C, in a proportion of 0.4 g of composition per g of hair.

After rinsing and drying, 4 lock portions, weighing 150 milligrams, are sampled from each lock, and are each mounted on a support.

Each support is then moved perpendicularly towards 2 stops: the maximum force required to continue the movement is recorded.

The mean force corresponding to the mean of the 4 maximum forces measured is calculated.

The greater the bending, the more resistant/tonic is the hair.

The following results are obtained:

| | Mean bending force (in N) |
|---|---|
| A (invention) | 0.0329 |
| B (comparative) | 0.0258 |
| C (comparative) | 0.0257 |

The composition A results in a bending force greater than that of the compositions B and C, which reflects a better bending resistance of the hair, and therefore a better tonicity.

In conclusion, only the composition A according to the invention makes it possible to produce a feel that is soft while at the same time remaining clean, and also allows the hair to retain tonicity.

### EXAMPLE 3

The cosmetic compositions below comprising the following ingredients (amounts expressed as % by weight of active material AM, unless otherwise indicated) are prepared:

| Ingredients | Invention A | Comparative A′ | |
|---|---|---|---|
| Cetearyl alcohol | 6% | 6% | |
| Mixture of myristyl stearate and myri- | 0.5% | 0.5% | |
| styl palmitate (INCI: CETYL ESTERS) | | | |
| Silicone emulsion as prepared in Ex- | 2% (0.8% AM of | — | |
| ample 1 (silicone + aminosilicone + | silicone + 0.2% AM | | |
| nonionic surfactants) | of aminosilicone) | | |
| Dimethicone ((Xiameter PMX-200 Sili- | — | 0.8% | am |
| cone Fluid) | | | |
| Amodimethicone (DC2-8566 Amino | — | 0.2% | am |
| Fluid) | | | |
| Non-ionic surfactants (laureth-11 + | — | 0.176% | am |
| PEG-100 stearate + steareth-10 + laureth- | | | |
| 3) | | | |
| Dicetyldimethylammonium chloride | 1% AM | 1% | AM |
| (VARISOFT 432 CG from Evonik) | | | |
| Cetyltrimethylammonium chloride | 1% AM | 1% | AM |
| (DEHYQUART A OR from BASF) | | | |

| | | |
|---|---|---|
| Preservative, fragrance | qs | qs |
| Water | qs 100% | qs 100% |

The compositions A and A′ were applied to pads of natural hair trimmings weighing 100 mg, previously washed with a standard shampoo and rinsed, in a proportion of 1 ml of composition per pad of hair.

After rinsing and drying, the silicon (Si) deposition has been measured, after the hair has been ground and homogenized, via the measurement of elemental Silicon by X-ray fluorescence spectrometry using a spectrometer S8 TIGER WDXRF from BRUKER (Wavelength Dispersion) XRF system.

The following results are obtained (in micrograms/gram of hair)

| | Total Si (μg/g hair) |
|---|---|
| A (invention) | 166 |
| A′ (comparative) | 554 |

It is noted that the invention composition A makes it possible to deposit amounts of silicone moderate in comparison with comparative composition A′.

As excessively large deposits of silicone result in an unclean greasy feel (loaded or charged touch), this is unappreciated by consumers.

With the invention composition, the deposit of silicone is thinner, and the feel (touch) is more natural and more clean (clean feel).

1.-17. (canceled)

18. A cosmetic composition comprising:
(i) at least one cationic surfactant of formula (Ia): [chemical structure]
wherein:
R5 and R6, which may be identical or different, represent a linear or branched, saturated or unsaturated aliphatic group comprising from 8 to 30 carbon atoms, wherein the aliphatic group optionally comprises at least one heteroatom; or an aromatic group;
R7 and R8, which may be identical or different, represent a linear or branched aliphatic group comprising from 1 to 6 carbon atoms;
Y⁻ is an anion;
(ii) at least one cationic surfactant other than the at least one cationic surfactant of formula (Ia), and
(iii) an oil-in-water emulsion having a particle size D50 of less than 350 nm, and comprising:
a silicone mixture comprising (i) a polydialkylsiloxane comprising trialkylsilyl end groups, having a viscosity at 25° C. ranging from 40 000 to 100 000 mPa·s and (ii) an aminosilicone having a viscosity at 25° C. ranging from 1000 to 15 000 mPa·s and an amine number ranging from 2 to 10 mg of KOH per gram of aminosilicone;
a surfactant mixture comprising one or more non-ionic surfactants, said mixture having an HLB ranging from 10 to 16, and
water.

19. The composition according to claim 18, comprising at least one cationic surfactant of formula (Ia) wherein:
R5 represents a saturated linear C8-C30 alkyl group, or a (C12-C22)alkylamido(C2-C6)alkyl group; and/or
R6 represents a saturated linear C8-C30 alkyl group, or a (C12-C22)alkyl acetate group; and/or
R7 and R8, which may be identical or different, represent a saturated linear alkyl group comprising from 1 to 6 carbon atoms.

20. The composition according to claim 18, comprising at least one cationic surfactant of formula (Ia) chosen from di(C8-C30)alkyldimethylammonium salts.

21. The composition according to claim 20, wherein the di(C8-C30)alkyldimethylammonium salts are chosen from distearyldimethylammonium halides, dicetyldimethylammonium halides, benzyldimethylstearylammonium halides, stearamidopropyldimethyl(myristyl acetate)ammonium halides, di(C8-C30)alkyldimethylammonium (C1-C4)alkyl sulfates, or mixtures thereof.

22. The composition according claim 18, wherein the at least one surfactant of formula (Ia) is present in a total amount ranging from 0.1° A to 10% by weight, relative to the total weight of the composition.

23. The composition according to claim 18, wherein the at least one cationic surfactant different from the cationic surfactants of formula (Ia) is chosen from the cationic surfactants of formula (Ib): [chemical structure]
wherein:
R1 represents a linear or branched, saturated or unsaturated aliphatic group comprising from 8 to 30 carbon atoms, wherein the aliphatic group optionally comprises one or more heteroatom,
R2 to R4, which may be identical or different, represent a linear or branched, saturated or unsaturated aliphatic group comprising from 1 to 6 carbon atoms; and
X⁻ is an anion chosen from halides, phosphates, acetates, lactates, (C1-C4)alkyl sulfates, (C1-C4)alkyl-, or (C1-C4)alkylarylsulfonates.

24. The composition according to claim 23, wherein the at least one cationic surfactant of formula (Ib) is chosen from (C8-C30)alkyltrimethylammonium salts.

25. The composition according to claim 24, wherein the (C8-C30)alkyltrimethylammonium salts are chosen from cetyltrimethylammonium salts, behenyltrimethylammonium salts, palm itylam idopropyltrimethylammonium salts, (C8-C30)alkyltrimethylammonium halides, (C8-C30)alkyltrimethylammonium (C1-C4)alkyl sulfates, or mixtures thereof.

26. The composition according to claim 18, wherein the at least one surfactant different from the cationic surfactants of formula (Ia) is present in a total amount ranging from 0.1% to 10% by weight, relative to the total weight of the composition.

27. The composition according to claim 23, wherein the at least one surfactant of formula (Ib) is present in a total amount ranging from 0.1° A to 10% by weight, relative to the total weight of the composition.

28. The composition according to claim 18, wherein the silicone mixture comprises one or more polydialkylsiloxanes comprising trialkylsilyl end groups of formula (I):
R′3SiO(R′2SiO)pSiR′3 ,
wherein:
R′, which may be identical or different, is a monovalent hydrocarbon-based radical having from 1 to 18 carbon atoms, and
p is an integer ranging from 500 to 2000;
having a viscosity ranging from 40 000 to 100 000 mPa s at 25° C.

29. The composition according to claim 18, wherein the silicone mixture comprises one or more aminosilicones of formula (II): XR₂Si(OSiAR)ₙ(OSiR₂)ₘOSiR₂X ,
wherein:
R, which may be identical or different, is a monovalent hydrocarbon-based radical having from 1 to 18 carbon atoms,
X, which may be identical or different, represents R or a hydroxyl (OH) or a C1-C6 0 alkoxy group;
A is an amino radical of formula —R¹−[NR²—R³—]ₓNR²₂, or the protonated form of this amino radical, with
R¹ representing a C1-C6 alkylene radical,
R², which may be identical or different, is a hydrogen atom or a C1-C4 alkyl radical,
R³ is a C1-C6 alkylene radical,
x is 0 or 1;
m and n are integers such that m+n ranges from 50 to 1000;
having a viscosity at 25° C. ranging from 1000 to 15 000 mPa·s, and/or an amine number ranging from 2 to 10 mg of KOH per gram of aminosilicone.

30. The composition according to claim 18, comprising (i) one or more polydialkylsiloxanes comprising trialkylsilyl end groups, having a viscosity, at 25° C., ranging from 40 000 to 100 000 mPa·s, in an amount of from 70% to 90% by weight, and (ii) one or more aminosilicones having a viscosity, at 25° C., ranging from 1000 to 15000 mPa·s and an amine number ranging from 2 to 10 mg of KOH per gram of aminosilicone, in an amount of from 10% to 30% by weight, relative to the total weight of the silicone mixture.

31. The composition according to claim 18, wherein the surfactant mixture comprises one or more non-ionic surfactants chosen from:
(i) (poly)oxyalkylenated fatty alcohols;
(ii) (poly)oxyalkylenated (C8-C32)alkyl phenyl ethers;
(iii) polyoxyalkylenated esters of C8-C32 fatty acids and of sorbitan; and
(iv) polyoxyethylenated esters of C8-C32 fatty acids.

32. The composition according to claim 18, wherein the oil-in-water emulsion comprises:
a surfactant mixture in a total amount ranging from 5% to 15% by weight, relative to the total weight of the emulsion; and/or
the non-ionic surfactant(s) in a total amount ranging from 5% to 15% by weight, relative to the total weight of the emulsion;
the silicone mixture in a total amount ranging from 40% to 60% by weight, relative to the total weight of the emulsion; and/or
the polydialkylsiloxane(s) comprising trialkylsilyl end groups, in a total amount ranging from 35% to 45% by weight, relative to the total weight of the emulsion; and/or
the aminosilicone(s) in a total amount ranging from 5% to 15% by weight, relative to the total weight of the emulsion; and/or
water in a total amount ranging from 25% to 50% by weight, relative to the total weight of the emulsion.

33. The composition according to claim 18, wherein the oil-in-water emulsion has a particle size D50 of between 100 and 300 nm.

34. The composition according to claim 18, wherein the oil-in-water emulsion is present in a total amount ranging from 0.1% to 10% by weight, relative to the total weight of the composition.

35. The composition according claim 18, wherein the composition comprises at least one alcohol, of structure R—OH wherein R denotes a linear or branched, saturated or unsaturated C8-C30 alkyl or alkenyl group, R optionally substituted with one or more hydroxyl groups.

36. A cosmetic process for treating keratin materials, wherein the process comprises;
applying to the keratin materials of a composition comprising:
(i) at least one cationic surfactant of formula (Ia): [chemical structure]
wherein:
R5 and R6, which may be identical or different, represent a linear or branched, saturated or unsaturated aliphatic group comprising from 8 to 30 carbon atoms, wherein the aliphatic group optionally comprises at least one heteroatom; or an aromatic group;
R7 and R8, which may be identical or different, represent a linear or branched aliphatic group comprising from 1 to 6 carbon atoms;
Y⁻ is an anion;
(ii) at least one cationic surfactant other than the at least one cationic surfactant of formula (Ia), and
(iii) an oil-in-water emulsion having a particle size D50 of less than 350 nm, and comprising:
a silicone mixture comprising (i) a polydialkylsiloxane comprising trialkylsilyl end groups, having a viscosity at 25° C. ranging from 40 000 to 100 000 mPa·s and (ii) an aminosilicone having a viscosity at 25° C. ranging from 1000 to 15 000 mPa·s and an amine number ranging from 2 to 10 mg of KOH per gram of aminosilicone;
a surfactant mixture comprising one or more non-ionic surfactants, said mixture having an HLB ranging from 10 to 16, and
water; and
optionally followed by a leave-on time and/or by a rinsing step and/or by a drying step.
